(19) United States
(12) Patent Application Publication
(10) Pub. No.: US 2015/0055089 A1
(43) Pub. Date: Feb. 26, 2015

(54) OPHTHALMIC PHOTOGRAPHING APPARATUS

(71) Applicant: NIDEK CO., LTD., Gamagori (JP)

(72) Inventors: Hideki AONO, Gamagori-shi (JP); Yukihiro HIGUCHI, Toyota-shi (JP)

(73) Assignee: NIDEK CO., LTD., Gamagori (JP)

(21) Appl. No.: 14/321,049

(22) Filed: Jul. 1, 2014

(30) Foreign Application Priority Data
Jul. 2, 2013 (JP) 2013-139067

(51) Int. Cl. A61B 3/10; A61B 3/00

(52) U.S. Cl. CPC A61B 3/102; A61B 3/0025
USPC 351/206

(57) ABSTRACT

An ophthalmic photographing apparatus including an OCT optical system for acquiring an OCT signal of a subject eye; an optical scanner for scanning the subject eye by the measurement light applied to the subject eye; a scan controller for controlling driving of the optical scanner and operate first scan control of respectively performing one scan by measurement light with respect to each of plural scan lines and second scan control of performing plural scans by measurement light with respect to each of the plural scan lines; and an image processor for acquiring the OCT signal in each scan line based on an output signal from the OCT optical system and perform composite processing on a plurality of the OCT signals of each scan line acquired by the second scan control using the OCT signal of each scan line acquired by the first scan control as a template.

### TECHNICAL FIELD

The present invention relates to an ophthalmic photographing apparatus for acquiring a tomographic image of a subject eye by optical coherence tomography, or an ophthalmic image processing program executed in an ophthalmic image processing apparatus for processing a tomographic image of a subject eye acquired by the ophthalmic photographing apparatus.

### BACKGROUND ART

An apparatus for imaging a tomographic image in a tissue (for example, a fundus or an anterior eye part) of an eye using optical coherence tomography (OCT) has been known. This apparatus scans a subject eye by measurement light using an optical scanner, and acquires the tomographic image. The obtained tomographic image is used in evaluation of a state of the eye (see JP-A-2010-110392).

Such an apparatus acquires an additional average image based on plural tomographic images obtained in the same scan line in order to average noise components included in the tomographic images. The additional average image is acquired by, for example, adding luminance values in each of the pixels in plural tomographic images on substantially the same site and obtaining an average value of the luminance values.

Also, a technique for correcting a misalignment by parallel translation and rotational transfer between plural tomographic images in the same scan line is performed in order to correct a misalignment between each of the tomographic images due to a misalignment of an eye.

In the case of performing plural scans by measurement light with respect to plural scan lines and obtaining an additional average image in each of the scan lines, after plural scans in one scan line are completed, a shift to plural scans in the next scan line is made.

### SUMMARY

Incidentally, as the number of scan lines, the number of acquired tomographic images in each of the scan lines, etc. are larger, the tomographic images are susceptible to involuntary eye movement or small movement of an eye by face movement etc. with time. For example, when small movement of an eye occurs between different scan lines, it is difficult to compare the tomographic images. Three-dimensional OCT data acquired with the influence of small movement of an eye included may have a shape different from the original shape of a subject eye since a positional relation of the eye with respect to the apparatus changes between the scan lines.

In view of the problem described above, one technical problem of the invention is to provide an ophthalmic photographing apparatus capable of acquiring good tomographic images with respect to plural scan lines.

In order to solve the problem described above, the invention is characterized by including the following configuration.

An ophthalmic photographing apparatus comprising:

an OCT optical system configured to acquire an OCT signal of a subject eye using interference between reference light and measurement light applied to the subject eye;

an optical scanner configured to scan the subject eye by the measurement light applied to the subject eye;

a scan controller configured to control driving of the optical scanner and operate first scan control of respectively performing one scan by measurement light with respect to each of plural scan lines and second scan control of performing plural scans by measurement light with respect to each of the plural scan lines; and

an image processor configured to acquire the OCT signal in each scan line by the scan controller based on an output signal from the OCT optical system and perform composite processing on a plurality of the OCT signals of each scan line acquired by the second scan control using the OCT signal of each scan line acquired by the first scan control as a template.

A computer-readable storage medium storing an ophthalmic image processing program executed by an ophthalmic image processing apparatus for processing a tomographic image of a subject eye acquired by ophthalmic optical coherence tomography, the ophthalmic optical coherence tomography comprising: an OCT optical system configured to acquire a tomographic image of the subject eye using interference between reference light and measurement light applied to the subject eye, an optical scanner configured to control the subject eye by the measurement light applied to the subject eye, and a scan controller configured to control driving of the optical scanner, the scan controller configured to operate first scan control of respectively performing one scan by measurement light with respect to each of plural scan lines and second scan control of performing plural scans by measurement light with respect to each of the plural scan lines, the ophthalmic image processing program executed by a processor of the ophthalmic image processing apparatus causing the ophthalmic image processing apparatus to execute:

acquiring a tomographic image in each scan line by the scan controller based on the OCT signal acquired by the OCT optical system; and

performing composite processing on a plurality of tomographic images of each scan line acquired by the second scan control using the tomographic image of each scan line acquired by the first scan control as a template image.

### BRIEF DESCRIPTION OF THE DRAWINGS

FIG. 1 is a schematic configuration diagram describing a configuration of an ophthalmic photographing apparatus according to the present embodiment.

FIG. 2 is a diagram showing one example of a display screen displayed on a display part 75.

FIG. 3 is a flowchart showing one example of first scan control and second scan control.

FIG. 4 is a schematic diagram describing one example of the first scan control and the second scan control.

### DESCRIPTION OF ILLUSTRATIVE EMBODIMENTS

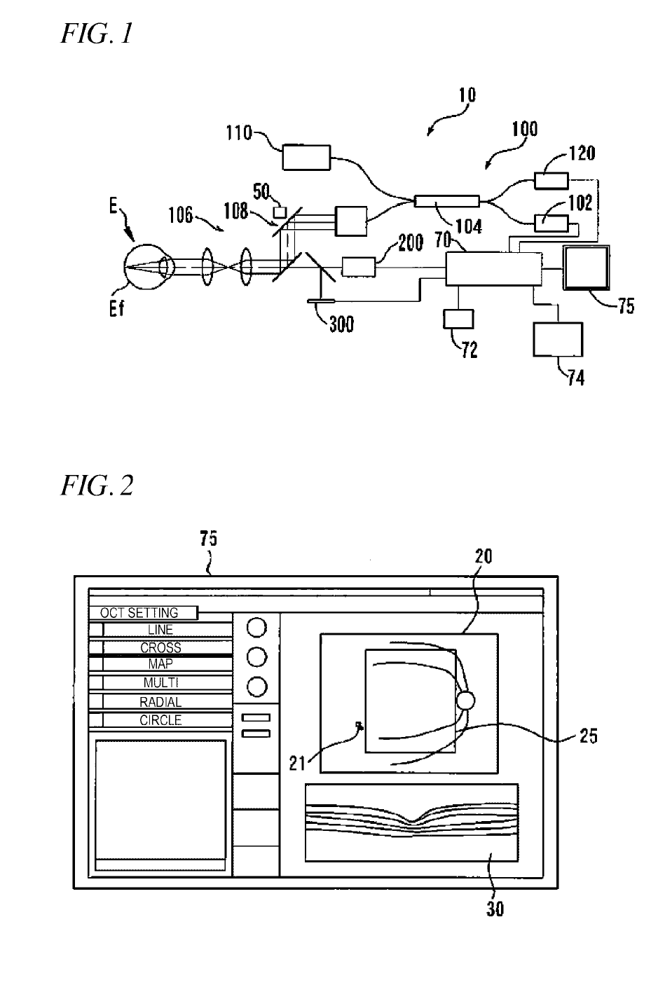

The present embodiment will hereinafter be described based on the drawings. FIG. 1 is a schematic configuration diagram describing a configuration of an ophthalmic photographing apparatus according to the present embodiment. In the following description, the ophthalmic photographing apparatus will be described by taking a fundus photographing apparatus for photographing a fundus of a subject eye as an example. Of course, the ophthalmic photographing apparatus is not limited to the fundus photographing apparatus, and includes, for example, an anterior eye photographing apparatus for photographing an anterior eye of a subject eye or an apparatus for photographing the whole subject eye.

A schematic configuration of an ophthalmic photographing apparatus 10 according to the embodiment will be described with reference to FIG. 1. The ophthalmic photographing apparatus 10 of the embodiment mainly includes an OCT optical system 100, an observation optical system 200, a fixation target projection unit 300, and a control part 70.

### <OCT Optical System>

The OCT optical system 100 is an optical coherence optical system for acquiring a tomographic image of a tissue (for example, a fundus Ef) of a subject eye E, and includes a configuration of optical coherence tomography (OCT). Concretely, the OCT optical system 100 mainly includes a measurement light source 102, a coupler (optical splitter) 104, a measurement optical system 106, a reference optical system 110, and a detector (light receiving element) 120.

More specifically, the coupler (optical splitter) 104 splits light emitted from the measurement light source 102 into an optical path of the measurement optical system 106 and an optical path of the reference optical system 110. The measurement optical system 106 guides measurement light to the fundus Ef of the eye E. The reference optical system 110 generates reference light. The OCT optical system 100 synthesizes the reference light and the measurement light reflected by the fundus Ef. The detector 120 (light receiving element) receives the synthesized light.

The OCT optical system 100 includes an irradiation position change unit (for example, an optical scanner 108, the fixation target projection unit 300) for changing an irradiation position of measurement light on the fundus Ef in order to change an imaging position on the fundus Ef. The control part 70 controls operation of the irradiation position change unit based on set imaging position information, and acquires a tomographic image based on a light receiving signal from the detector 120.

The detector 120 (light receiving element) detects a state of interference between measurement light and reference light. In the case of Fourier domain OCT, spectral intensity of interference light is detected by the detector 120 and a depth profile (A scan signal) in a predetermined range is acquired by Fourier transformation to spectral intensity data. Various OCTs can be adopted in the ophthalmic photographing apparatus 10. For example, any of Spectral-domain OCT (SD-OCT), Swept-source OCT (SS-OCT) and Time-domain OCT (TD-OCT) may be adopted in the ophthalmic photographing apparatus 10.

The optical scanner 108 scans a fundus of a subject eye by light emitted from a measurement light source. For example, the optical scanner 108 scans the fundus by measurement light two-dimensionally (in an XY direction (transverse direction)). The optical scanner 108 is arranged in a position conjugate to a pupil. The optical scanner 108 is, for example, two galvano-mirrors, and its reflection angle is freely adjusted by a driving mechanism 50.

Accordingly, a direction of reflection (travel) of luminous flux emitted from the light source 102 is changed, and the fundus is scanned in any direction. Accordingly, an imaging position on the fundus Ef is changed. The optical scanner 108 could be configured to deflect light. For example, reflective minors (a galvano-mirror, a polygon mirror and a resonant scanner) and an acousto-optical element (AOM) for changing a direction of travel (deflection) of light are used.

The reference optical system 110 generates reference light. As described above, the reference light is synthesized to reflected light acquired by reflection of the measurement light on the fundus Ef. The reference optical system 110 may be a Michelson type or a Mach-Zehnder type. The reference optical system 110 is formed of, for example, a reflective optical system (for example, a reference minor), and again returns light from the coupler 104 to the coupler 104 by reflecting the light by the reflective optical system, and guides the light to the detector 120. As another example, the reference optical system 110 is formed of a transmitting optical system (for example, an optical fiber), and does not return light from the coupler 104, and guides the light to the detector 120 by transmitting the light.

The reference optical system 110 has a configuration of changing an optical path length difference between measurement light and reference light by moving an optical member in a reference optical path. For example, a reference mirror is moved in an optical axis direction. The configuration of changing the optical path length difference may be arranged in a measurement optical path of the measurement optical system 106.

### <Front Observation Optical System>

The front observation optical system (front image observation device) 200 is formed in order to obtain a front image of the fundus Ef. The observation optical system 200 includes, for example, an optical scanner for two-dimensionally scanning the fundus by measurement light (for example, infrared light) emitted from a light source, and a second light receiving element for receiving fundus reflected light through a confocal opening arranged in a position conjugate to the fundus, and has an apparatus configuration of the so-called ophthalmic scanning laser ophthalmoscope (SLO).

In addition, a configuration of the observation optical system 200 may be a configuration of the so-called fundus camera type. Also, the OCT optical system 100 may share the observation optical system 200. In other words, the front image may be acquired using data forming a tomographic image obtained two-dimensionally (for example, an integration image of three-dimensional tomographic images in a depth direction, an integration value of spectral data in each XY position, luminance data in each XY position in a certain depth direction or a retina surface layer image).

### <Fixation Target Projection Unit>

The fixation target projection unit 300 has an optical system for guiding a sight line direction of the eye E. The projection unit 300 has a fixation target presented to the eye E, and can guide the eye E in plural directions.

For example, the fixation target projection unit 300 has a visible light source for emitting visible light, and two-dimensionally changes a presentation position of an eye target. Accordingly, the sight line direction is changed, with the result that an imaging site is changed. For example, when a fixation target is presented from the same direction as a photographic optical axis, the center of a fundus is set as the imaging site. Also, when the fixation target is presented upwardly with respect to the photographic optical axis, an upper portion of the fundus is set as the imaging site. In other words, the photographic site is changed according to a position of the eye target with respect to the photographic optical axis.

The fixation target projection unit 300 includes various configurations such as a configuration of adjusting a fixation position by a lighting position of LEDs arranged in a matrix state or a configuration of scanning by light from a light source using an optical scanner and also adjusting a fixation position by lighting control of the light source. Also, the projection unit 300 may be an internal fixation lamp type or an external fixation lamp type.

### <Control Part>

The control part 70 includes a CPU (processor), RAM, ROM, etc. The CPU of the control part 70 performs control of the ophthalmic photographing apparatus 10. Various pieces of information are temporarily stored in the RAM. Initial values, various programs, etc. for controlling operation of the ophthalmic photographing apparatus 10 are stored in the ROM of the control part 70.

Nonvolatile memory (hereinafter abbreviated as memory) 72, a manipulation part 74, a display part 75, etc. are electrically connected to the control part 70. The memory 72 is a non-temporary storage medium capable of holding the contents of storage even when supply of a power source is broken. For example, a hard disk drive, flash ROM, or USB memory detachably attached to the ophthalmic photographing apparatus 10 can be used as the memory 72. A photography control program for controlling photography of a front image or a tomographic image by the ophthalmic photographing apparatus 10, and an image processing program for processing the front image or the tomographic image are stored in the memory 72. Also, various pieces of information about photography, for example, information about photography positions of photographed two-dimensional tomographic images, three-dimensional images, front images or tomographic images are stored in the memory 72. Various manipulation instructions by an examiner are inputted to the manipulation part 74.

The manipulation part 74 outputs signals according to the inputted manipulation instructions to the control part 70. As the manipulation part 74, for example, at least any of a mouse, a joy stick, a keyboard, a touch panel, etc. may be used. The display part 75 may be a display mounted in a body of the ophthalmic photographing apparatus 10 or a display connected to the body. A display of a personal computer (hereinafter called a “PC”) may be used. Plural displays may be used together. Various images including a tomographic image and a front image photographed by the ophthalmic photographing apparatus 10 are displayed on the display part 75.

In addition, the control part 70 may be constructed of plural control parts (that is, plural processors). For example, the control part 70 of the ophthalmic photographing apparatus 10 may be constructed of a setting control part formed in the PC and an operation control part for controlling operation of the OCT optical system 100 etc. In this case, for example, the setting control part of the PC can set an imaging position etc. of a tomographic image based on operation of the manipulation part connected to the PC and instruct the operation control part on the set contents. The operation control part can control photography operation by each configuration of the ophthalmic photographing apparatus 10 according to instructions from the setting control part. Also, any of the operation control part and the setting control part may perform processing for generating (acquiring) an image based on a light receiving signal.

For example, the control part 70 acquires a tomographic image by image processing based on a light receiving signal outputted from the detector 120 of the OCT optical system 100. The control part 70 acquires a front image based on a light receiving signal outputted from a light receiving element of the observation optical system 200. The control part 70 controls the fixation target projection unit 300 to change a fixation position.

For example, the control part 70 controls a display screen of the display part 75. An acquired fundus image is outputted to the display part 75 as a still image or a moving image, and is further stored in the memory 72. The control part 70 controls each of the members of the OCT optical system 100, the observation optical system 200 and the fixation target projection unit 300 based on a manipulation signal outputted from the manipulation part 74.

### <Control Operation>

Control operation in the apparatus including the above configuration will be described. An examiner instructs a subject to gaze at a fixation target of the fixation target projection unit 300. An anterior eye observation image photographed by a camera for anterior eye observation (not shown) is displayed on the display part 75. Hence, the examiner performs an alignment manipulation so as to position a measurement optical axis in the center of a pupil of an anterior eye part.

The control part 70 controls driving of the optical scanner 108 and scans a fundus in a predetermined direction by measurement light. The control part 70 forms a tomographic image by acquiring a light receiving signal corresponding to a predetermined scan region from an output signal outputted from the detector 120. The control part 70 controls the OCT optical system 100 and acquires a tomographic image. The control part 70 controls the observation optical system 200 and acquires a fundus front image. The control part 70 acquires the tomographic image by the OCT optical system 100 and acquires the fundus front image by the observation optical system 200 at any time.

FIG. 2 is a diagram showing one example of a display screen displayed on the display part 75. The control part 70 displays a tomographic image 30, scan pattern display 25 and a front image 20 acquired by the observation optical system 200 on the display part 75. The scan pattern display 25 is an index representing a measurement position (acquisition position) of the tomographic image on the front image 20. The scan pattern display 25 is electrically displayed on the front image on the display part 75.

The control part 70 displays a pointer 21 (for example, a cross mark, a dot mark or a pen mark) on the display part 75. The control part 70 moves the pointer 21 based on a manipulation signal from the manipulation part 74.

The embodiment is constructed so that a photographing condition can be set by manipulating the manipulation part 74 (for example, a drag manipulation or a click manipulation) with the pointer 21 positioned on the front image 20. The pointer 21 is used for specifying any position on the display part 75. An examiner may move the scan pattern display 25 with respect to the front image by performing a movement manipulation (for example, a drag manipulation) using the manipulation part 74.

### <Setting of Scan Line>

The case of setting a raster scan as a scan pattern will hereinafter be described by way of example. In addition, the scan pattern is preset in any shape base on a manipulation of the examiner. For example, the scan pattern is selected from plural prepared scan patterns.

After a tomographic image and a front image are displayed on the same screen, the examiner sets a position of the tomographic image to be photographed from the front image on the display part 75. The front image 20 and the tomographic image 30 are preferably displayed as a live moving image. In the case of the raster scan, a part of the tomographic image is displayed as the moving image.

### <Raster Scan>

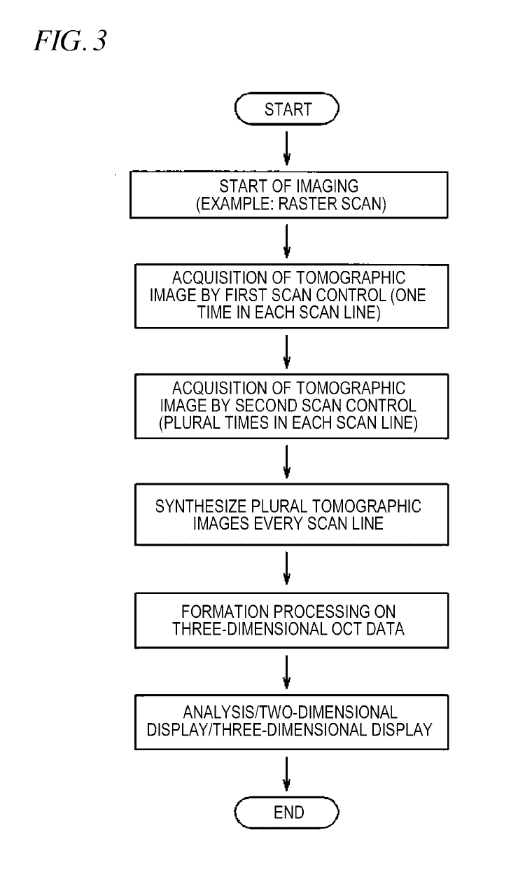

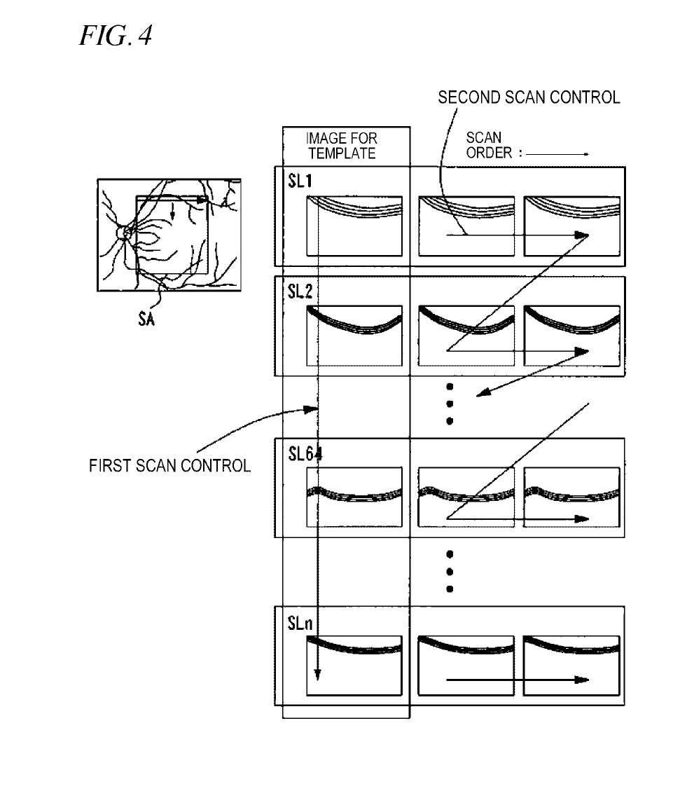

The control part 70 acquires a tomographic image in a scan position corresponding to a preset scan pattern by controlling the optical scanner 108. A raster scan is a pattern in which the fundus Ef is scanned in a rectangular shape by measurement light (see FIGS. 3 and 4).

The raster scan is used as, for example, a scan for obtaining an analysis map. The analysis map shows, for example, two-dimensional distribution of thickness in a fundus tissue. In the raster scan, for example, a preset scan region (for example, a rectangular region) is rasterized by the measurement light. As a result, a tomographic image in each scan line of the inside of the scan region (for example, the rectangular region) is acquired.

As scan conditions of the raster scan, for example, line widths (distance from a starting point to an ending point) in a horizontal scanning direction and a vertical scanning direction, a scan speed, a spacing between scan lines, and the number of scan lines are preset. Of course, the scan conditions of the raster scan may be configured to be set freely.

More specifically, the control part 70 forms a tomographic image along the horizontal scanning direction by scanning in the horizontal scanning direction by measurement light in a scan line (the first line) set as a start position. Next, the control part 70 forms a tomographic image along the horizontal scanning direction by scanning in the horizontal scanning direction by measurement light in a different scan line in the vertical scanning direction. As described above, the tomographic images are obtained respectively with respect to N lines different mutually. The tomographic image of the inside of the scan region can be acquired by decreasing each scan spacing in the vertical scanning direction. The scan region is formed by different scan lines in the vertical scanning direction.

In the following description, the case of setting the vertical scanning direction as a Y direction (up-and-down) and setting the horizontal scanning direction as an X direction (right-and-left direction) is described by way of example, but it is not limited to this case. For example, the vertical scanning direction may be the X direction and the horizontal scanning direction may be the Y direction.

In scan control in the vertical scanning direction, a scan position may be changed sequentially from top to bottom, or the scan position may be changed sequentially from bottom to top. Also, the scan position may be changed sequentially from the center to the periphery. Also, an interlace method may be used as the raster scan.

### <First Scan Control and Second Scan Control>

FIG. 3 is a flowchart showing one example of first scan control and second scan control. FIG. 4 is a schematic diagram describing one example of the first scan control and the second scan control. When a trigger signal of the start of photography from the manipulation part 74 is inputted, the control part 70 acquires tomographic images by the first scan control and the second scan control different mutually. The first scan control and the second scan control are performed in, for example, a preset scan region.

The acquired tomographic images are stored in the memory 72 as the still images. For example, the control part 70 changes the number of acquired tomographic images in each scan line between the first scan control and the second scan control.

In the first scan control, the control part 70 sequentially performs a one-by-one scan by measurement light respectively with respect to each different scan line in the vertical scanning direction. The control part 70 generates a tomographic image in each scan line, and stores the generated tomographic image in the memory 72. Since the number of scans in each scan line is limited in the first scan control, the time necessary to acquire the tomographic images in the whole region can be shortened. The first scan control is used as scanning for obtaining, for example, a template image in image synthesis (for example, image additional averaging).

In the second scan control, the control part 70 performs plural scans by measurement light with respect to each different scan line in the vertical scanning direction. The control part 70 generates plural tomographic images in each scan line, and stores the generated tomographic images in the memory 72. The second scan control is used as scanning for obtaining, for example, plural object images in image synthesis (for example, image additional averaging). The object image is used as an image synthesized to the template image.

One example of the first scan control and the second scan control is shown below.

### <Example of First Scan Control>

As illustrated in FIG. 4, when a trigger signal of the start of photography from the manipulation part 74 is inputted, the control part 70 obtains a tomographic image in each scan line by first scan control in order to acquire a template image. Then, the control part 70 scans in the horizontal scanning direction by measurement light in a first scan line SL1 by controlling the optical scanner 108. The detector 120 receives light of interference between reference light and fundus reflected light obtained by the measurement light, and outputs a light receiving signal to the control part 70. The control part 70 generates a tomographic image corresponding to the first scan line SL1 based on an output signal from the detector 120.

After the completion of the scan in the first scan line SL1, the control part 70 scans in the horizontal scanning direction by measurement light in a second scan line SL2 by controlling the optical scanner 108. Then, the control part 70 generates a tomographic image corresponding to the second scan line SL2. Similarly, the control part 70 generates tomographic images corresponding to each of the scan lines by performing the scan by measurement light in a third scan line SL3, . . . , an (n−1)th scan line SLn−1 and an nth scan line SLn, respectively. That is, in the first scan control in the embodiment, the scan is performed one by one with respect to each scan line. After the completion of the scan in the last scan line, the first scan control is completed.

The control part 70 acquires tomographic images corresponding to the scan lines SLi (i=1 to n) in a scan range SA based on the output signals from the detector 120. The control part 70 associates each of the acquired tomographic images with each of the scan lines and stores each of the tomographic images in the memory 72. Each of the tomographic images is captured (taken) as a still image, and is stored in the memory 72.

### <Example of Second Scan Control>

After the completion of the first scan control, the control part 70 shifts to second scan control. The shift from the first scan control to the second scan control may be made automatically or based on a trigger signal from the manipulation part 74. Automatic control is effective in that, for example, time can be shortened, and manual control is effective in that a state of a subject can be checked before the start of the second scan control requiring time relatively.

In the second scan control, the control part 70 performs plural scans by measurement light respectively with respect to each scan line by controlling the optical scanner 108. The control part 70 respectively acquires plural tomographic images corresponding to each scan line.

For example, the control part 70 performs plural scans in the horizontal scanning direction by measurement light in the first scan line SL1. That is, after the completion of the first scan from a starting point to an ending point in the first scan line SL1, the control part 70 again returns a scan position of the measurement light to the starting point in the first scan line SL1, and again scans in the first scan line SL1.

The control part 70 generates plural tomographic images corresponding to the first scan lines SL1 based on an output signal from the detector 120. In the second scan control, plural tomographic images are acquired by plural scans in the same scan position. The control part 70 scans in the first scan line SL1, for example, until tomographic images of a preset number of frames are obtained.

After the completion of the plural scans in the first scan line SL1, the control part 70 performs plural scans in the horizontal scanning direction by measurement light in the second scan line SL2 by controlling the optical scanner 108. The control part 70 generates plural tomographic images corresponding to the second scan lines SL2. The control part 70 scans in the second scan line SL2, for example, until tomographic images of a preset number of frames are obtained.

Similarly, the control part 70 generates tomographic images corresponding to each of the scan lines by performing the plural scans by measurement light in the third scan line SL3, . . . , the (n−1)th scan line SLn−1 and the nth scan line SLn, respectively. That is, in the second scan control, the plural scans are performed with respect to each scan line. After the completion of the scan in the last scan line, the second scan control is completed.

The control part 70 stores tomographic images corresponding to the scan lines SLi (i=1 to n) in the scan range SA in the memory 72. Each of the tomographic images is captured as a still image, and is associated with each of the scan lines and is stored.

### <Tracking>

The control part 70 may track measurement light with respect to a transverse position on a set fundus by controlling driving of the optical scanner 108 based on an eye image acquired by the observation optical system 200.

For example, the control part 70 may correct a scan position by detecting a misalignment between a live moving image acquired by the observation optical system 200 and a previously acquired still image (reference image) by image processing and controlling driving of the optical scanner 108 based on its detection result. In the case of correcting the scan position, a misalignment is preferably detected including parallel translation and rotational transfer of a fundus. The control part 70 adjusts the scan position in the optical scanner 108 in order to correct the detected misalignment.

Actuation of tracking is particularly advantageous as measures against the misalignment in the case of acquiring plural tomographic images in the same position in the second scan control. The control part 70 acquires a still image acting as the basis for tracking, for example, before the second scan control. Of course, tracking may be actuated in both of the first scan control and the second scan control.

### <Composite Processing on Plural Tomographic Images>

Plural tomographic images obtained by the second scan control are synthesized. The control part 70, for example, performs composite processing (for example, additional average processing) of plural tomographic images acquired in the same scan line. As a result, a composite image is acquired every scan line. In addition, according to the additional average processing, speckle noise is reduced and the tomographic image with a good contrast is acquired.

The control part 70 obtains image composite data (for example, additional average data) by synthesizing plural tomographic images (hereinafter called second tomographic images) acquired by the second scan control using a tomographic image (hereinafter called a first tomographic image) acquired by the first scan control as a template image. The obtained image composite data is stored in the memory 72.

In addition, in the case of synthesizing images, the control part 70 may detect a misalignment between the first tomographic image and the second tomographic image by image processing and perform alignment (matching) between the tomographic images based on its detection result by image processing. The misalignment between the tomographic images is corrected by such processing. In addition, see, for example, JP-A-2010-110392 for an alignment technique (of course, a misalignment correction technique is not limited to this technique). As a method for detecting the misalignment between the images, at least any of various image processing techniques (a method using various correlation functions, a method using Fourier transformation, and a method based on matching of a feature point) can be used.

Since the plural second tomographic images are acquired with respect to the same scan line, processing of matching between the first tomographic image and each of the tomographic images included in the plural second tomographic images is performed. Accordingly, the misalignment between the plural tomographic images acquired with respect to the same scan line is corrected based on the first tomographic image.

For example, the control part 70 performs matching between the first tomographic image obtained in the first scan line SL1 and the plural second tomographic images obtained in the first scan line SL1, and synthesizes the first tomographic image and the plural second tomographic images. Accordingly, the control part 70 acquires image composite data in the first scan line SL1. Similarly, the control part 70 can acquire image composite data in the other scan lines.

The obtained image composite data is stored in the memory 74. The image composite data may be the additional average image itself, and also be luminance information (luminance information obtained by adding luminance of each image) serving as a basis for the additional average image.

When the additional average data is obtained as the image composite data, the control part 70 may acquire the additional average data based on plural tomographic images, for example, by using absolute values (A scan signal after imaging) of a real component and an imaginary component of depth information forming a tomographic image. Also, the control part 70 may acquire the additional average data by using real and imaginary components in Z space serving as a basis for each tomographic image. The control part 70 may obtain first additional average data using a signal of a real component and also obtain second additional average data using a signal of an imaginary component. The control part 70 may acquire the additional average data based on plural tomographic images by synthesizing first and second additional average data.

### <Generation of Three-Dimensional Data>

In the embodiment, the control part 70 performs formation processing on three-dimensional OCT data based on a total of N image composite data obtained in each scan line. As the formation processing on the three-dimensional OCT data, for example, a publicly known technique of processing on interpolation between adjacent tomographic images is used. The control part 70 stores the obtained three-dimensional data in the memory 74.

As one advantage by the control described above, the tomographic image of each scan line is acquired in a shorter time than the case of sequentially acquiring plural tomographic images of each line since the first scan control acquires the tomographic image of each scan line one by one. Thus, since the tomographic image is resistant to being influenced by involuntary eye movement or small movement by face movement, there is a low possibility of misaligning the tomographic images between the different scan lines. For example, first three-dimensional OCT data (tomographic image set) close to a shape of a fundus of a subject eye can be obtained.

Consequently, three-dimensional composite OCT data close to the shape of the fundus of the subject eye can be obtained by matching and synthesizing second three-dimensional OCT data (plural object images) obtained by the second scan control every scan line using the tomographic image acquired by the first scan control as a template. For example, in the case of additional average data, three-dimensional OCT data close to the actual shape of the fundus of the subject eye and having good image quality of the tomographic image of each scan line can be obtained.

### <Analysis of Three-Dimensional OCT Data>

A use example of three-dimensional composite OCT data is shown below.

For example, the control part 70 may analyze three-dimensional composite OCT data by image processing and obtain an analysis result. The control part 70 may output the obtained analysis result to the display part 75. The analysis result includes, for example, thickness information about at least one of a retina layer of a subject eye, and size information (for example, a ratio of C (cup) to D (disk) of an optic nerve head) about a characteristic site of a fundus.

The control part 70 may output the obtained analysis result to the display part 75 as an analysis map. The analysis map includes, for example, a retina thickness map or a choroid layer thickness map.

The retina thickness map may be a map indicating two-dimensional distribution of retina thickness of the subject eye and is, for example, color-coded according to layer thickness. The retina thickness map includes a thickness map, a comparison map, a deviation map, an examination date comparison thickness difference map, etc.

In the three-dimensional composite OCT data, a misalignment of an analysis result between each line is small since first three-dimensional OCT data close to an actual shape of a fundus of the subject eye is used as a template. For example, in the case of three-dimensional OCT data added and averaged, the three-dimensional OCT data is analyzed with high accuracy and a good analysis result is acquired since the misalignment of the analysis result between each line is small and further image quality of the tomographic image of each scan line is good.

In addition, a technique for outputting a result of analysis on three-dimensional OCT data is not limited to the analysis map and, for example, the control part 70 may output the obtained analysis result to the display part 75 as an analysis chart (for example, a G chart, an S/I chart, an ETDRS chart or a TSNIT chart) in which two-dimensional distribution of a retina layer is obtained as the average every region.

<Acquisition of Tomographic Image from Three-Dimensional OCT Data>

For example, the control part 70 may extract (acquire) a tomographic image from three-dimensional OCT data acquired previously. The control part 70 may output the extracted tomographic image to the display part 75.

Here, the control part 70, for example, generates an OCT front image which is a front image of a fundus from three-dimensional OCT data and outputs the OCT front image to the display part 75. According to such a front image, a contrast of a small blood vessel can be improved.

The OCT front image is obtained by, for example, integrating signal strength distribution of a depth direction in a Z direction in each XY position of three-dimensional OCT data (the so-called integrated image). Of course, the OCT front image may be acquired by processing different from the integrating processing. Also, the OCT front image may be, for example, a retina surface layer OCT image or a C scan image indicating signal strength distribution in a certain depth position.

In addition, the control part 70 may acquire a front image based on a phase signal of a spectral signal serving as a basis for the three-dimensional OCT data acquired as described above. For example, the control part 70 generates a front image according to the number of zero cross points in an interference signal (for example, JP-A-2011-215134). Luminance unevenness may occur depending on a tilt of a fundus in the front image of this method. According to the embodiment thus, a good template can be obtained, with the result that, for example, a front image with small luminance unevenness is acquired and also a contrast of a small blood vessel is improved.

The control part 70 superimposes a setting line for setting an acquisition position on an OCT front image. The control part 70 accepts a manipulation signal from the manipulation part 74 and adjusts a display position of the setting line. The control part 70 extracts a tomographic image corresponding to the display position of the setting line from three-dimensional OCT data and outputs the tomographic image to the display part 75. In addition, the setting line may be a direction (for example, an orthogonal direction or an oblique direction) different from a scanning direction of measurement light.

In three-dimensional composite OCT data, a misalignment of a tomographic image between each line is small since first three-dimensional OCT data close to an actual shape of a fundus of a subject eye is used as a template. Consequently, even in the case of displaying the tomographic image in the direction different from the scanning direction of measurement light, the good tomographic image is displayed.

For example, in the case of three-dimensional OCT data added and averaged, the misalignment of the analysis result between each line is small and further, image quality of the tomographic image forming the three-dimensional OCT data is good. As a result, even after the three-dimensional OCT data is acquired, an examiner can check the tomographic image in any position with good image quality. Consequently, for example, it is useful in identifying a lesion from the tomographic image.

In addition, the fundus front image is not limited to the OCT front image. The fundus front image may be, for example, a fundus front image (hereinafter described as a second front image) obtained by at least any of a fundus camera and SLO. Here, the control part 70 obtains a correspondence relation between the three-dimensional OCT data and the second front image obtained. For example, the control part 70 may obtain the correspondence relation by matching the above OCT front image with the second front image by image processing.

By way of example, the control part 70 superimposes a setting line for setting an acquisition position on the second front image. The control part 70 accepts a manipulation signal from the manipulation part 74 and adjusts a display position of the setting line. The control part 70 acquires a tomographic image corresponding to the display position of the setting line from three-dimensional OCT data and outputs the tomographic image to the display part 75.

In addition, a use example of the three-dimensional OCT data is not limited to the above. For example, the control part 70 may construct a three-dimensional OCT graphic image based on the three-dimensional OCT data and output the graphic image to the display part 75.

### Modified Example

In addition, in the case of obtaining an additional average image using a template image obtained by the first scan control, the control part 70 may add plural object images to a specified number of added images in real time while tomographic images are obtained in each scan line. Also, after the tomographic images are acquired in each scan line, addition operation may be performed. The specified number of added images can be changed freely.

The control part 70 may determine whether or not the object image is proper to a template image, and choose the object image based on a determination result. For example, the control part 70 may eliminate the object image from an object of image composition when a correlation value obtained between the template image and the object image does not satisfy an acceptable range. When the correlation value is small, there is a high possibility that the template image greatly differs from the object image in a photography region due to, for example, involuntary eye movement or a misalignment between the apparatus and an eye, but its possibility can be decreased by the processing described above.

In addition, in the case of determining that the object image is not proper, the control part 70 may determine whether or not the object image is proper to a template image of another scan line (for example, an adjacent scan line), and associate the object image with an image acquired in the scan line in the case of determining that the object image is proper as a result. Accordingly, time taken to acquire the image can be made more efficient.

In addition, determination as to whether or not the object image is proper is not limited to this. For example, the control part 70 may eliminate a front image in which a detected misalignment amount exceeds an acceptable range from an object of addition processing.

The embodiment is not limited to map photography. For example, the embodiment can be applied to a scan pattern in which plural mutually different scan lines are arranged. In the first scan control, the control part 70 sequentially performs one scan by measurement light respectively with respect to each scan line. In the second scan control, the control part 70 performs plural scans by measurement light with respect to each scan line.

An example of the scan pattern in which plural mutually different scan lines are arranged includes a wide range of scans of cross, radial, multi lines, etc. In the cross scan, a cross pattern in which plural scan lines are orthogonal longitudinally and transversely is used. In the radial scan, a radial pattern in which plural scan lines are radially arranged is used. In the multi line, a pattern in which plural scan lines separated mutually are arranged is used.

In addition, the control part 70 may perform Fourier transformation of an output signal (spectral signal) from the OCT optical system 100 and acquire a signal after performing the Fourier transformation as an OCT signal. The control part 70 performs composite processing on plural OCT signals of each scan line acquired by the second scan control using an OCT signal of each scan line acquired by the first scan control as a template. As the OCT signal, for example, tomographic image data after Fourier transformation, phase information data after Fourier transformation or signal strength data in Z space after Fourier transformation are acquired.

In addition, in the above, additional average processing is illustrated as image composite processing based on plural tomographic images, but the image composite processing is not limited to this additional average processing. As the image composite processing, for example, the control part 70 may perform super-resolution processing based on plural tomographic images. Accordingly, three-dimensional OCT data close to an actual shape of a fundus of a subject eye and having good image quality of the tomographic image of each scan line can be obtained.

Further, the embodiment is not limited to the image composite processing, and can be applied to image composite processing based on plural tomographic images. As the composite processing, for example, the control part 70 may acquire a blood flow measurement image (Doppler OCT image) by measuring a change (for example, a phase change or a strength change) between signals in plural OCT signals based on the plural OCT signals in addition to the image composite processing. Also, the control part 70 may acquire an image indicating polarization properties of a subject eye by measuring polarization components (S polarization, P polarization) in plural OCT signals based on the plural OCT signals. That is, the embodiment can also be applied to OCT such as the Doppler OCT or the polarization sensitive OCT.

In the case of obtaining the Doppler OCT image, for example, the control part 70 could acquire an OCT signal in each scan line by the first scan control and the second scan control described above. In this case, the control part 70 scans in each scan line by the number of scans (for example, two to four times) set in order to obtain the Doppler image in the second scan control. The control part 70 acquires the Doppler OCT image with respect to each scan line based on at least two OCT signals acquired with timing different temporally. The control part 70, for example, can acquire two-dimensional distribution information about a blood flow of a fundus based on the Doppler OCT image with respect to each scan line. In addition, see, for example, BIOMEDICAL OPTICS EXPRESS P803-P821, Izatt. et. “Automated non-rigid registration and mosaicing for robust imaging of distinct retinal capillary beds using speckle variance optical coherence tomography” 2013 OSA 1 Jun. 2013|Vol. 4, No. 6 for the details of a technique for acquiring the Doppler OCT image.

A change between signals in the case of obtaining the Doppler OCT image is well detected since, for example, a proper template is acquired by applying control of the embodiment to acquisition of the Doppler OCT image.

In addition, in the example described above, in the case of synthesizing plural object images obtained by the second scan control using a template image obtained by the first scan control, the control part 70 may synthesize the plural object images including the template image, and may also synthesize the plural object images using the template image as only a template of a misalignment correction.

In addition, a choice of an object image using the template image described above can be made smoothly by performing the first scan control previously and performing the second scan control later. However, the order of the first scan control and the second scan control is not particularly limited.

In addition, the control part 70 may change scan conditions such as a scan speed, a scan width, a scan line spacing, or exposure time of a light receiving element in addition to the number of acquired images in each line between the first scan control and the second scan control. In addition, the control part 70, for example, may acquire a composite image by performing scans in a common scan region in the first scan control and the second scan control. In this case, at least any of a part of the scan region, the scan width, the scan line spacing and the exposure time of the detector (light receiving element) may be changed in the first scan control and the second scan control. In addition, the control part 70 may make settings in the same scan position, the same scan width or the same scan line spacing between the first scan control and the second scan control.

Also, the control part 70 may repeat scan control of sequentially performing one scan by measurement light respectively with respect to each scan line different mutually plural times. Here, the control part 70, for example, may synthesize plural tomographic images in each scan line obtained by another scan control plural times using the tomographic image of each scan line obtained by any of the plural scan controls as a template.

In addition, the first scan control and the second scan control may be performed by different scanners. Measurement light sources may differ in the first scan control and the second scan control. In the case of the different scanners, the first scan control and the second scan control may be performed simultaneously.

Also in such control, for example, in the case of an additional average image, three-dimensional OCT data close to a shape of a fundus of a subject eye and having good image quality of the tomographic image of each scan line can be obtained.

In addition, the control part 70 may generate three-dimensional OCT data based on a tomographic image of each scan line and acquire an OCT front image from the generated three-dimensional OCT data. The control part 70 performs at least the first scan control of sequentially performing one scan by measurement light respectively with respect to each scan line different mutually. The control part 70 generates an OCT front image (first OCT front image) from the three-dimensional OCT data acquired by the first scan control. The control part 70 generates plural OCT front images (second OCT front images) from plural three-dimensional OCT data acquired by another scan control. The control part 70 may correct a misalignment between the plural second OCT front images using the first OCT front image as a template.

What is claimed is:

1. An ophthalmic photographing apparatus comprising:
an OCT optical system configured to acquire an OCT signal of a subject eye using interference between reference light and measurement light applied to the subject eye;
an optical scanner configured to scan the subject eye by the measurement light applied to the subject eye;
a scan controller configured to control driving of the optical scanner and operate first scan control of respectively performing one scan by measurement light with respect to each of plural scan lines and second scan control of performing plural scans by measurement light with respect to each of the plural scan lines; and
an image processor configured to acquire the OCT signal in each scan line by the scan controller based on an output signal from the OCT optical system and perform composite processing on a plurality of the OCT signals of each scan line acquired by the second scan control using the OCT signal of each scan line acquired by the first scan control as a template.

2. The ophthalmic photographing apparatus as claimed in claim 1, wherein, as the composite processing, the image processor adds and averages the plurality of tomographic images acquired by the second scan control using the OCT signal acquired by the first scan control as a template.

3. The ophthalmic photographing apparatus as claimed in claim 1, wherein in a case of setting a scan pattern in which plural scan lines having relations equal in a first scanning direction and different in a second scanning direction are arranged adjacently, the image processor forms three-dimensional OCT data based on the tomographic image of each scan line in which composite processing is performed.

4. The ophthalmic photographing apparatus as claimed in claim 1, wherein the scan controller is configured to operate first scan control of sequentially performing one scan by measurement light in each scan line and second scan control of sequentially performing plural scans in each scan line in the case of performing plural scans by measurement light with respect to each of the plural scan lines.

5. The ophthalmic photographing apparatus as claimed in claim 1, wherein the scan controller continuously performs the first scan control and the second scan control.

6. The ophthalmic photographing apparatus as claimed in claim 1, wherein the image processor further determines whether or not an OCT signal acquired by the second scan control is proper as an object signal with respect to the OCT signal acquired by the first scan control as a template, and makes a choice based on the determination result.

7. The ophthalmic photographing apparatus as claimed in claim 1, wherein, as the composite processing, the image processor measures a change between signals in the plurality of OCT signals acquired by the second scan control with respect to the OCT signal acquired by the first scan control as a template.

8. The ophthalmic photographing apparatus as claimed in claim 1, wherein the image processor performs Fourier transformation of an output signal from the OCT optical system and acquires a signal after performing the Fourier transformation as the OCT signal.

9. The ophthalmic photographing apparatus as claimed in claim 1, wherein the scan controller performs first scan control and second scan control by a raster scan.

10. The ophthalmic photographing apparatus as claimed in claim 1, wherein the image processor acquires first OCT three-dimensional data based on an OCT signal of each scan line acquired by the first scan control, and acquires second OCT three-dimensional data based on an OCT signal of each scan line acquired by the second scan control.

11. The ophthalmic photographing apparatus as claimed in claim 1, wherein the scan controller changes a scan condition between first scan control and second scan control.

12. The ophthalmic photographing apparatus as claimed in claim 1, wherein the scan controller changes at least any of a scan speed, a scan width, a scan line spacing, and exposure time of a light receiving element formed in the OCT optical system between the first scan control and the second scan control.

13. The ophthalmic photographing apparatus as claimed in claim 1, wherein the scan controller performs the first scan control and then performs the second scan control later.

14. The ophthalmic photographing apparatus as claimed in claim 1, wherein
the image processor:
generates first OCT three-dimensional data based on the OCT signal of each scan line acquired by the first scan control;
acquires a first OCT front image based on the generated first OCT three-dimensional data;
generates a plurality of second OCT three-dimensional data based on the OCT signals of each scan line acquired by the second scan control; and
acquires a plurality of second OCT front images based on the generated second OCT three-dimensional data.

15. The ophthalmic photographing apparatus as claimed in claim 14, wherein the image processor corrects a misalignment of the plurality of second OCT front images using the first OCT front image.

16. A computer-readable storage medium for storing an ophthalmic image processing program executed in an ophthalmic image processing apparatus for processing a tomographic image of a subject eye acquired by ophthalmic optical coherence tomography,
the ophthalmic optical coherence tomography comprising:
an OCT optical system configured to acquire a tomographic image of the subject eye using interference between reference light and measurement light applied to the subject eye,
an optical scanner configured to scan the subject eye by the measurement light applied to the subject eye, and
a scan controller configured to control driving of the optical scanner, the scan controller capable of operating first scan control of respectively performing one scan by measurement light with respect to each of plural scan lines and second scan control of performing plural scans by measurement light with respect to each of the plural scan lines,
the ophthalmic image processing program executed by a processor of the ophthalmic image processing apparatus causing the ophthalmic image processing apparatus to execute performing composite processing of plural OCT signals of each scan line acquired by the second scan control using an OCT signal of each scan line acquired by the first scan control as a template image by being executed by a processor of the ophthalmic image processing apparatus.
